(19) United States
(12) Patent Application Publication
(10) Pub. No.: US 2025/0210204 A1
(43) Pub. Date: Jun. 26, 2025

(54) SYSTEM AND METHOD FOR PREDICTIVE RISK ASSESSMENT AND INTERVENTION

(71) Applicant: Morgan State University, Baltimore, MD (US)

(72) Inventors: Lorece Edwards, Windsor Mill, MD (US); Lawrence Brown, Baltimore, MD (US); Sabriya Dennis, Abingdon, MD (US); Ian Lindong, Baltimore, MD (US)

(21) Appl. No.: 19/079,829

(22) Filed: Mar. 14, 2025

Related U.S. Application Data
(63) Continuation-in-part of application No. 17/942,393, filed on Sep. 12, 2022, now abandoned.
Application No. 17/942,393 is a continuation of application No. 16/668,860, filed on Oct. 30, 2019, now abandoned.
(60) Provisional application No. 62/752,530, filed on Oct. 30, 2018.

(51) Int. Cl. G16H 50/30; G06N 5/02; G16H 10/20; G16H 10/60; G16H 20/00

(52) U.S. Cl. CPC G16H 50/30; G06N 5/02; G16H 10/20; G16H 10/60; G16H 20/00

(57) ABSTRACT

A computer-implemented system and method for predictive risk assessment and intervention includes a risk assessment unit that processes survey data through an integrated database architecture. The system employs machine learning algorithms to analyze relationships between community and personal risk factors, maintaining real-time correlation coefficients and reliability metrics in a geospatial database. Machine learning algorithms may process factor analysis results to generate risk prediction quotients and determine intervention thresholds. The system automatically recalibrates based on intervention outcomes, continuously updating statistical relationships while preserving geographic and demographic associations. The database architecture coordinates multiple specialized components, enabling real-time analysis of risk patterns and automated generation of evidence-based intervention recommendations. The machine learning implementation continuously improves prediction accuracy through automated learning, while maintaining statistical validity through reliability calculations and correlation analysis.

### CROSS REFERENCE TO RELATED APPLICATIONS

This application is a continuation-in-part of U.S. patent application Ser. No. 17/942,393 titled “SYSTEM AND METHOD FOR PREDICTIVE RISK ASSESSMENT AND INTERVENTION,” filed with the United States Patent & Trademark Office on Sep. 12, 2022, which application is a continuation of U.S. patent application Ser. No. 16/668,860 titled “SYSTEM AND METHOD FOR PREDICTIVE RISK ASSESSMENT AND INTERVENTION,” filed with the United States Patent & Trademark Office on Oct. 30, 2019, which application is based upon and claims the benefit of U.S. Provisional Application No. 62/752,530 titled “Perceived Risk Hierarchy Methodology,” filed with the United States Patent & Trademark Office on Oct. 30, 2018, the specifications of which are incorporated herein by reference in their entireties.

### GOVERNMENT RIGHTS STATEMENT

This invention was made with Government support under contract number SP020188-01 awarded by the United States Substance Abuse and Mental Health Services Administration (SAMHSA). The Government may have certain rights in the invention.

### FIELD OF THE INVENTION

This invention is directed to computer-implemented systems and methods for processing complex statistical relationships between risk factors in real-time through an integrated database architecture and machine learning implementation. More particularly, the invention relates to technical improvements in computer functionality for maintaining dynamic correlations between community and personal risk factors, automatically generating statistically validated risk assessments, and recalibrating predictive models based on intervention outcomes through specialized data processing components and machine learning algorithms. The invention provides specific technological improvements to computer-based risk assessment systems through automated statistical validation methods, real-time geographic pattern analysis, and machine learning-driven intervention planning that maintains reliability metrics across multiple data components while processing continuous updates to risk factor relationships.

### BACKGROUND OF THE INVENTION

A number of populations throughout the United States, and in fact throughout the world, face various dangers that are brought about by their own behaviors and activities. The likelihood of their participation in those danger-prone behaviors and activities has been found to relate at least partially to their perception of various risks that they face in their daily lives. Further, those perceptions of the risks that they face in their lives are shaped by their own cultural and social environments and interactions.

For example, it has been found that when approaching emerging adults with respect to HIV-risk behaviors and perceptions, even with the best of incentives, emerging adults in certain urban populations were not testing for HIV and did not perceive communicable disease as a concern, and did not consider themselves at risk of contracting a communicable disease. It has been found that an individual's perception of these and other risks shapes that individuals' activities, and in fact perceptions about certain risks may tend to induce high-risk behaviors in certain individuals. For instance, it has been found that youth and emerging adults' perception of health risk or severity is attenuated by what they perceive as more imminent and immediate risks, such as matters of personal safety, danger, and perceived survival expectations. As a result, the individual's perceived risk hierarchy can influence the individual's likelihood of engaging in negative behaviors, such as: (1) indifference to sexually transmitted infections (STI), HIV and HCV prevention screening that have been made significantly more convenient and accessible compared to traditional testing and screening modalities; (2) a low perception of STI, HIV and HCV risk despite engagement in high-risk behaviors, and (3) knowingly engaging in these behaviors despite knowledge of perils and potential detrimental outcomes. Additionally, it has been found that the individual's survival expectations are linked to a range of authentic concerns (e.g., internalized symptoms such as depression, anxiety, fear, and hopelessness) and externalizing behaviors, such as aggression. The inventors herein have found that certain populations live each day on high alert for perceived threats and are in a constant state of mobilizing for fight, flight, or suspense as they anticipate the next assault, such that it becomes less likely for them to concentrate, learn, recall, do well in school, consider employment, perceive a future orientation, and delay immediate gratification. As a result, they are more likely to absorb and trivialize health and other risks that are otherwise real and can have deleterious impact. However, intervention by health and other professionals may likewise modify the individual's perceptions regarding risk, and in turn modify their behavior to move away from high-risk behaviors. Nonetheless, given the difficulty in reaching large numbers of at-risk populations, there is only limited success in achieving behavior modification in this way.

As an individual's perceived risk hierarchy may impact the likelihood that they will engage in certain dangerous behaviors, and as intervention tools do exist (such as counseling, education, etc.) that can help to adjust the individual's perceptions of the risks they face in daily life, it would be advantageous to provide systems and methods by which data may be collected from larger portions of at-risk populations to evaluate their perceived risk hierarchy, and by which intervention tools may be automatically suggested for use by intervention service providers (e.g., counselors, medical professionals, etc.) and their successes tracked in order to reduce the likelihood that such individuals will engage in the negative activities.

Unfortunately, prior attempts to assess and mitigate behavioral risks have been limited by an inability to process complex relationships between multiple risk factors in real-time, a lack of sophisticated data storage systems capable of tracking geographic and demographic patterns, and absence of automated statistical validation methods for risk assessments, and manual intervention planning without quantitative effectiveness metrics. Traditional risk assessment systems rely on static databases and basic statistical analysis, making them inadequate for processing the dynamic relationships between perceived risks and actual behavioral outcomes. These systems cannot effectively maintain real-time correlations between community and personal risk factors or automatically adjust risk assessments based on intervention outcomes. Likewise, these systems cannot generate statistically validated intervention recommendations or track geographic distribution patterns of risk indicators.

Furthermore, existing systems lack the machine learning capabilities necessary to process complex factor analysis results across multiple risk domains, calculate and continuously update path coefficients between variables, generate dynamic risk prediction quotients, or automatically recalibrate based on intervention effectiveness. The technical challenges of risk assessment are compounded by the need to maintain statistical reliability across multiple data components and process real-time updates while preserving data relationships, in addition to the needs to generate automated interventions based on validated correlations and track intervention outcomes across geographic and demographic segments.

These technical limitations have prevented effective large-scale risk assessment and intervention, particularly in urban environments where multiple risk factors interact in complex ways. Thus, there remains a need in the art for systems and methods capable of addressing these technical challenges through an integrated database architecture and sophisticated machine learning implementation that enables automated, real-time risk assessment and intervention planning.

### SUMMARY OF THE INVENTION

Disclosed herein is a system and method for predictive risk assessment and intervention that avoids one or more disadvantages of the prior art. A system is described herein having a computer-implemented predictive risk assessment unit that receives survey data from a remotely connected survey device. The survey data comprises information about the social and cultural environment of one or more members of a risk population to create a digital perceived risk hierarchy inventory for each surveyed population member, which may include data such as the member's perceptions of their social and environmental factors (e.g., police contact, community violence, experience with substance abuse, exposure to sexually transmitted diseases, etc.), the member's demographic data (e.g., age, gender, education level, etc.), and publicly available data associated with the member's geographic environment (e.g., local homelessness, drug treatment, arrests, etc.). The digital perceived risk hierarchy is transmitted from the remote survey device to the predictive risk assessment unit, where a risk determination engine analyzes each such perceived risk hierarchy inventory to generate a risk portfolio for each surveyed member of the population. The risk portfolio may include a risk predictive quotient profile for each such member that assigns a numeric value indicating a likelihood of that member engaging in certain negative activities or exhibiting negative behaviors, or in a composite of multiple negative activities or behaviors, a recommendation of interventions that are determined to reduce the likelihood of such member engaging in those negative activities and/or exhibiting those negative behaviors, and preferably a record of success and/or failure of various interventions in reducing that risk. At least a portion of the risk portfolio (including at least the recommendation of interventions) is then transmitted from the predictive risk assessment unit to one or more intervention partners who administer the interventions to the surveyed members, record the success or failure of such intervention in preventing the identified dangerous behavior, and transmit an intervention effectiveness report to the predictive risk assessment unit. The predictive risk assessment unit may then modify and recalibrate the survey instrument (and particularly weights assigned to various elements of the digital perceived risk hierarchy inventory) and the associated recommended intervention products and tools using various analytical methods to maximize the successes of interventions, particularly as evidenced in further intervention effectiveness reports received form intervention partners.

The system implements an integrated database architecture centered around a real-time geospatial database that coordinates multiple specialized data components. This database architecture maintains survey templates, individual risk portfolios, and risk factor relationships while preserving geographic and demographic associations. The system employs advanced statistical modeling techniques to process risk assessment data, including correlation coefficients that measure relationships between risk variables, reliability metrics that validate assessment consistency, and dynamic intervention thresholds that trigger automated recommendations. The database continuously updates these statistical measurements based on new survey responses and intervention outcomes, enabling real-time analysis of risk patterns across different geographic locations and demographic segments. This integrated approach allows the system to automatically adjust risk assessments and intervention recommendations while maintaining statistical reliability and data consistency across all components.

Systems and methods configured in accordance with certain aspects of the invention provide a snapshot of the cultural and social environment of the member of the risk population and provide an insight into the future possibility of that population member engaging in at-risk behaviors or activities. Thus, such systems and methods may provide strong indications of where intervention planning can be employed to mitigate such behaviors. Likewise, the results of actual interventions may suggest whether refinement of the survey or the analytic methods performed by the predictive risk assessment unit is necessary or warranted.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The numerous advantages of the present invention may be better understood by those skilled in the art by reference to the accompanying drawings in which:

FIG. 1 is a schematic view of a system for predictive risk assessment and intervention in accordance with certain aspects of an embodiment of the invention.

FIG. 2 shows a Confirmatory Factor Analysis demonstrating how five risk subscales (Community Disrepair, Community Violence, Community Crime, Housing Instability, and Violence Experience) form two main latent risk factor variables—Community Risk Factors and Personal Risk Factors. The statistical relationships between these variables are shown by beta coefficients (β), with values ranging from 0.58 to 0.86, indicating strong correlations. The two main risk factors have a correlation (r) of 0.63 between them.

FIG. 3 shows a Path Analysis showing how Personal Risk Projection (the outcome

variable) is influenced by Community Risk Factors, Social Adjustment, Police Peril Assessment, and Personal Risk Factors. The analysis shows multiple pathways of influence, with beta coefficients indicating the strength and direction of relationships. For example, Personal Risk Factors have a strong positive influence (β=0.34) on Personal Risk Projection.

FIG. 4 shows a Mediation Analysis examining how Police Peril Assessment mediates the relationship between Social Adjustment and Personal Risk Projection. It shows both direct effects (c=−0.34) and indirect effects through Police Peril Assessment, demonstrating how the relationship changes when accounting for this mediating variable.

FIG. 5 is a representation of a risk predictive quotient matrix for use with the system of FIG. 1.

FIG. 6 is a schematic flowchart of a method for predictive risk assessment and intervention in accordance with further aspects of an embodiment of the invention.

FIG. 7 is a schematic view of a computing device for use with the system of FIG. 1.

### DETAILED DESCRIPTION

The invention summarized above may be better understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

To the knowledge of the inventors herein, the combination of perception, risk, and hierarchy have not previously been addressed in a manner that may be used to automatically connect factors addressing a risk population member's environment (e.g., an urban environment), the resultant risks that they face based on the cultural and social environment characteristics of that environment, and the interventions that may best mitigate those risks. The systems and methods employed herein are based upon a finding that emerging adults prioritize risk within their own framework for survival and success. The priority risk should be acknowledged, addressed, and satisfied so that the emerging adults can proceed to practice prevention, observe proper practices and behaviors, focus on positive short-and long-term goals, increase academic performance and attain educational goals, as well as maintain a positive orientation going forward. As disclosed in greater detail below, the perceived risk hierarchy inventory of a surveyed emerging adult member of the risk population is analyzed at a predictive risk assessment unit to develop a behavior risk profile and assessment and based upon such behavior risk profile and assessment, automatically assign recommended intervention products that are determined to reduce the member's risk of engaging in harmful behavior. Such recommended intervention products (and preferably other elements of the risk population member's behavior risk profile and assessment) are transmitted to at least one intervention partner (e.g., educational institutions, community centers, hospitals, counselors, physician's offices, etc.) to allow that partner to use and track the success of intervention measures, and transmit back to the predictive risk assessment unit an intervention effectiveness report which may be used to further refine the automated analytical tools used to evaluate the member's risk of engaging in harmful behavior.

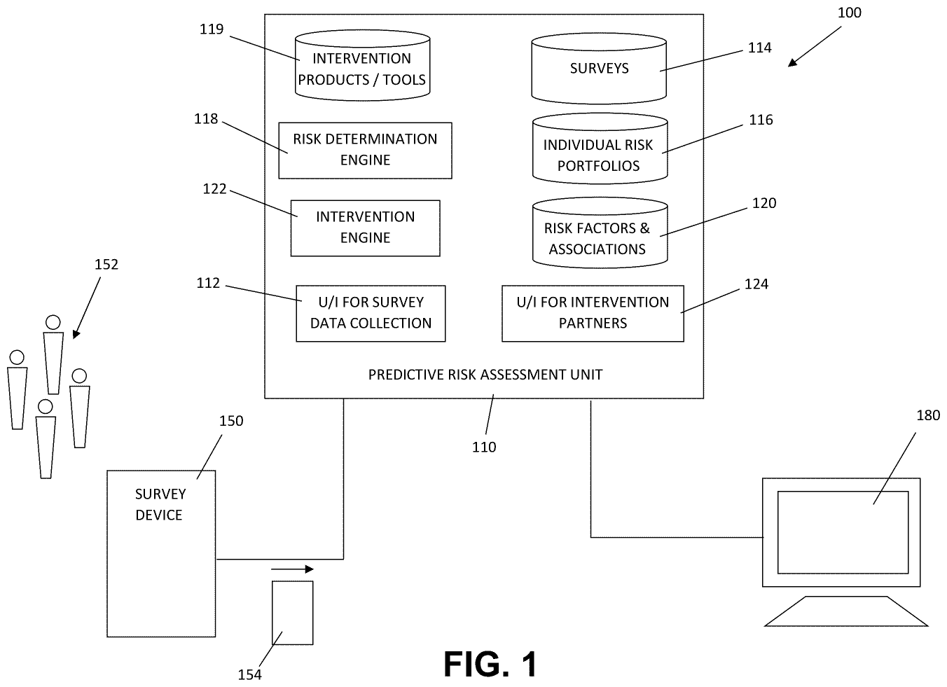

FIG. 1 shows an exemplary schematic representation of a system for predictive risk assessment and intervention (shown generally at 100) including a predictive risk assessment unit 110, one or more computer implemented remote survey devices 150 in remote data communication with predictive risk assessment unit 110, and one or more intervention partner computers 180 in data communication with predictive risk assessment unit 110. Predictive risk assessment unit 110 is preferably a hosted system that may, by way of non-limiting example, be hosted in a cloud processing environment accessible via a wide area data network such as the Internet.

Survey device 150 is preferably a remote computing device, such as a tablet, a laptop computer, a smartphone, or similarly configured readily portable computing device, configured for remote communication with predictive risk assessment unit 110. Survey device 150 is used to record responses from one or more members of risk population 152 at least relating to each such surveyed member's perceived risk hierarchy, and preferably also relating to certain demographic data relating to such surveyed member. Predictive risk assessment unit 110 preferably hosts a user interface 112 for survey data collection, which preferably receives a login request from survey device 150 and authenticates the user (e.g., through password entry or other such authentication methods as may be chosen by those skilled in the art) to predictive risk assessment unit 110. Predictive risk assessment unit 110 may receive a request from survey device 150 for a survey from survey database 114 and may transmit a digital survey to survey device 150 for administering to the member of risk population 152. Preferably, the digital survey includes survey questions that solicit the population member's perception of various cultural and social factors in their day-to-day environment, in addition to certain demographic data relating to that risk population member. The digital survey may thus collect experiential perception profile data, including (by way of non-limiting example) data indicative of the risk population member's perception of and exposure to police contact and negative community experiences (e.g., HIV/AIDS and other sexually transmitted diseases, mental health impairment, substance abuse, community violence, etc.). The digital survey may additionally collect individual demographic profile data, including (by way of non-limiting example) data indicative of the risk population member's employment status (e.g., unemployed, employed part-time, employed full-time), education level (e.g., high school, college, post-graduate education), zip code (or smaller geographic designation), voter registration status, age, gender, sexual orientation, and church attendance. Of course, other experiential perception profile data and demographic profile data may be included in the digital survey as may be preferable for a given risk population, which may be readily determined by persons skilled in the art.

The data collected by the digital survey may form a perceived risk hierarchy inventory 154 that may be transmitted from survey device 150 to predictive risk assessment unit 110 through user interface 112, and risk assessment unit 110 may generate and store in data memory an individual risk portfolio 116 for the surveyed risk population member 152 that includes the member's perceived risk hierarchy inventory 154 (i.e., their experiential perception profile data and demographic profile data), as discussed in detail below. In exemplary configurations, predictive risk assessment unit 110 may supplement the member's individual risk portfolio 116 with existing public data that may be geospatial in nature (e.g., publicly available community demographic data), which may be helpful to further determine interrelationships among various cultural and social factors that may affect a risk population member's likelihood of engaging in dangerous or high-risk behaviors.

Using statistical analytical methods, a risk determination engine 118 may analyze the surveyed member's experiential perception profile data and demographic profile data collected in their individual risk profile 116 and may generate a risk predictive quotient matrix 156 for a variety of risk segments for that risk population member 152. By way of non-limiting example, the risk segments may include sexual health factors (e.g., HIV and other sexually transmitted diseases and teen pregnancy), mental health factors (e.g., substance abuse, suicide and depression), and violence and injury factors (e.g., gun violence, domestic violence and child abuse). Once generated, the risk predictive quotient for that risk population member 152 may then be added to and stored with the member's individual risk portfolio 116.

The risk determination engine implements multiple advanced statistical modeling techniques to analyze risk factors and generate predictive assessments, which techniques enable concrete, real-world risk assessment and intervention outcomes. The system employs a two-stage statistical validation process to ensure accuracy and reliability of risk predictions. This two-stage process directly enables intervention partners to identify at-risk individuals before negative behaviors manifest, quantify specific risk levels across multiple dimensions, target interventions to highest-risk population segments, and track intervention effectiveness through measurable outcomes.

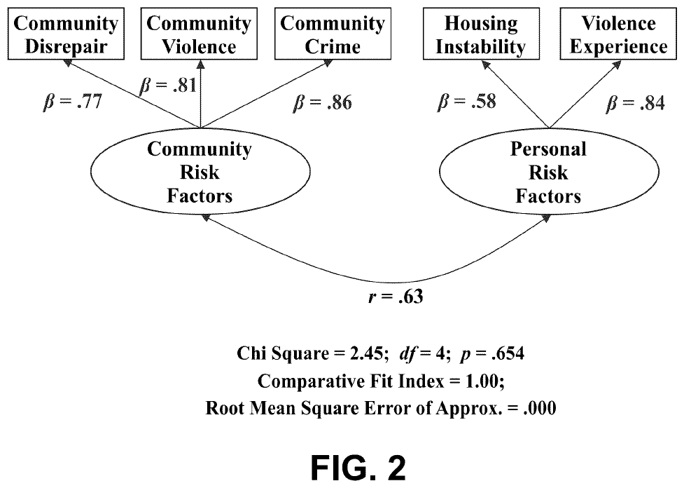

In a first stage (Factor Analysis and Model Validation) and with reference to FIG. 2, the system performs confirmatory factor analysis to identify and validate two primary latent risk variables-Community Risk Factors and Personal Risk Factors. This analysis quantifies the statistical relationships between five key risk subscales: Community Disrepair (β coefficient=0.77); Community Violence (β coefficient=0.81); Community Crime (β=0.86); Housing Instability (β coefficient=0.58); and Violence Experience (β coefficient =0.84). The strong beta coefficients demonstrate statistical validity of the underlying risk assessment model. The two primary risk factors maintain a correlation coefficient of 0.63, enabling the system to accurately predict relationships between community and personal risk elements.

Correlation coefficients represent statistical measurements of relationships between risk variables, calculated through confirmatory factor analysis to quantify the strength and direction of associations between community risk factors, personal risk factors, and intervention outcomes. These coefficients range in value from −1 to 1, with values closer to +1 indicating stronger relationships.

The confirmatory factor analysis of Community and Personal Risk Factors allows intervention partners to pinpoint specific environmental risk factors requiring immediate intervention, determine whether community-level or individual-level interventions will be most effective, and allocate intervention resources based on statistically-validated risk priorities.

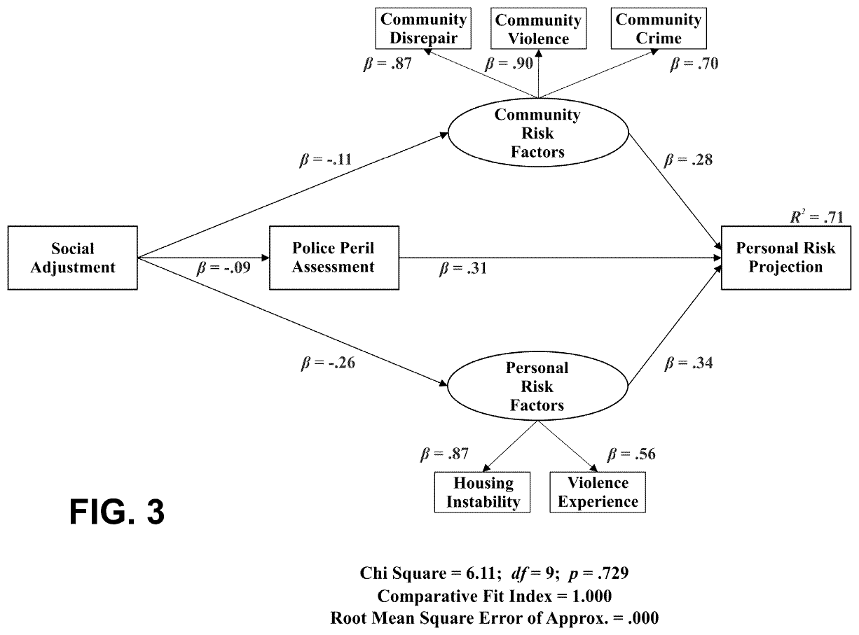

In a second stage (Path Analysis and Risk Projection) and with reference to FIG. 3, the predictive risk assessment engine employs path analysis modeling to determine how multiple risk factors influence Personal Risk Projection outcomes. The path analysis quantifies: Direct effects of Community Risk Factors (β=0.28); Mediating effects of Social Adjustment (β=−0.11); Impact of Police Peril Assessment (β=0.31); and Influence of Personal Risk Factors (β=0.34). This path analysis modeling enables the system to generate individualized risk portfolios with specific intervention recommendations and to automatically adjust intervention strategies based on measured success rates. Moreover, the path analysis modeling can provide early warning indicators when risk factors reach critical thresholds and can track risk reduction progress through quantifiable metrics.

As shown in Table 1 below, the system maintains internal reliability through reliability coefficients (i.e., Cronbach's alpha coefficients) calculated for each risk assessment subscale. The following Table 1 presents reliability coefficients for the Perceived Risk Hierarchy Theory (PRHT) Inventory Subscales. The reliability coefficients ensure intervention partners can consistently identify high-risk individuals across different populations, compare risk levels between different geographic areas, measure the effectiveness of different intervention approaches, and make evidence-based decisions about resource allocation. Reliability metrics, implemented through Cronbach's alpha coefficients, measure the internal consistency and statistical reliability of risk assessment subscales. These metrics validate the consistency of survey responses and risk assessments across multiple data points, with values above 0.7 generally indicating strong reliability for risk prediction purposes.

Table 1 shows strong reliability across most subscales, with coefficients ranging from 0.67 to 0.94. The overall PRHT Composite has a very strong reliability coefficient of 0.94.

TABLE 1
Reliability Coefficients for the Perceived Risk Hierarchy
Theory (PRHT) Inventory Subscales and Composite (N = 275)

| Subscale | Number of Items | Alpha Coefficient |
|---|---|---|
| Police Peril (PE) | 10 | .82 |
| Community Disrepair (CR) | 6 | .92 |
| Community Violence (CV) | 4 | .81 |
| Community Crime (CC) | 6 | .93 |
| Housing Instability (HI) | 6 | .85 |
| Violence Experience (VE) | 6 | .67 |
| Projected Risk (PR) | 12 | .94 |
| PRHT Composite | 50 | .94 |

The risk determination engine carries out real-time statistical processing by continuously updating a correlation matrix tracking relationships between all measured variables. Table 2 provides a correlation matrix showing relationships between all PRHT Inventory subscales and Social Adjustment Index values.

TABLE 2
Matrix of Bivariate Correlation Coefficients for PRHT Inventory Subscales
Scores and Social Adjustment Index Values (N = 275) (the index
was derived from six demographic items added to the PRHT Inventory)

| | Variable | | | | | | | |
|---|---|---|---|---|---|---|---|---|
| Variable | (A) | (B) | (C) | (D) | (E) | (F) | (G) | (H) |
| (A) Police Peril | | | | | | | | |
| (B) Community Disrepair | .32** | | | | | | | |
| (C) Community Violence | .24** | .62** | | | | | | |
| (D) Community Crime | | .21** | .63** | .69** | | | | |
| (E) Housing Instability | .09 | .14* | .19** | .20** | | | | |
| (F) Violence Experience | .16* | .20** | .30** | .28** | .48** | | | |
| (G) Projected Risk | .46** | .41** | .40** | .43** | .29** | .47** | | |
| (H) PRHT Composite | .42** | .77** | .79** | .81** | .44** | .49** | .49** | |
| (I) Social Adjustment | −.11 | −.17 | −.10 | −.11 | −.17 | −.22** | −.34** | −.24** |

*p < .05;
**p < .01

Notable correlations include strong relationships between Community Disrepair, Community Violence, and Community Crime (coefficients ranging from 0.62 to 0.69), and significant negative correlations between Social Adjustment and various risk measures. Such strong correlations between community risk factors (coefficients ranging from 0.62 to 0.69) enable the system to automatically adjust risk assessments as new data is received.

Table 3 below compares PRHT Inventory subscale scores between church attendees and non-attendees. The analysis shows significant differences in several subscales, with non-church attendees generally showing higher risk scores. For example, non-attendees scored significantly higher on Police Peril (70.54 vs 62.26) and Projected Risk (51.32 vs 38.94).

TABLE 3
Summary of Hotellings Multiple t-Test on PRHT
Inventory Subscales Scores by Church
Attendance (N = 264) (reflects all non-missing cases)

| | Church Attendance | | | | |
|---|---|---|---|---|---|
| | Yes | | No | | |
| | (n = 99) | | (n=165) | | |
| Subscale | X̅ | σ | X̅ | σ | F-ratio |
| Police Peril | 62.26 | 11.48 | 70.54 | 14.78 | 18.62** |
| Community Disrepair | 67.87 | 24.86 | 74.89 | 25.03 | 3.67* |
| Community Violence | 53.37 | 25.87 | 52.12 | 22.48 | 0.25 |
| Community Crime | 58.53 | 29.20 | 61.17 | 23.99 | 0.49 |
| Housing Instability | 26.24 | 14.77 | 31.04 | 17.47 | 4.74* |
| Violence Experience | 16.28 | 7.26 | 19.30 | 7.58 | 8.26** |
| Projected Risk | 38.94 | 21.97 | 51.32 | 21.52 | 17.92** |
| PRHT Composite^{††} | 46.39 | 12.56 | 51.32 | 13.88 | 2.81** |

*p < .05; **p < .01
^{††}Results based on Independent t-Test method

Table 4 below presents similar comparisons based on voter registration status. The analysis reveals some significant differences between registered and non-registered voters, particularly in Police Peril, Housing Instability, and Projected Risk subscales.

TABLE 4
Summary of Hotellings Multiple t-Test on PRHT
Inventory Subscales Scores by Voter
Registration (N = 264) (reflects all non-missing cases)

| | Registered Voter | | | | |
|---|---|---|---|---|---|
| | Yes | | No | | |
| | (n = 99) | | (n = 165) | | |
| Subscale | X̅ | σ | X̅ | σ | F-ratio |
| Police Peril | 65.22 | 13.21 | 69.98 | 14.51 | 6.20* |
| Community Disrepair | 70.09 | 24.63 | 74.13 | 25.19 | 1.20 |
| Community Violence | 54.37 | 25.32 | 51.47 | 21.79 | 0.93 |
| Community Crime | 61.06 | 26.68 | 59.07 | 25.14 | 0.49 |
| Housing Instability | 31.23 | 19.61 | 27.09 | 13.21 | 4.27* |
| Violence Experience | 17.97 | 7.97 | 18.53 | 7.26 | 0.84 |
| Projected Risk | 44.19 | 22.58 | 50.28 | 22.29 | 4.37* |
| PRHT Composite^{††} | 49.15 | 12.77 | 49.89 | 14.17 | 0.42 |

Hotellings T-Squared F-ratio = 4.14**
*p < .05; **p < .01
^{††}Results based on Independent t-Test method

The system further employs multivariate analysis of variance (MANOVA) to analyze how demographic factors impact risk scores across multiple dimensions. For example, Table 5 below shows a Multivariate Analysis of Variance comparing PRHT Inventory subscale scores across employment status (Unemployed, Part-Time, and Full-Time). The results demonstrate significant differences across employment categories, with unemployed individuals generally showing higher risk scores across multiple subscales. For example, employment status analysis reveals statistically significant differences in risk profiles, with Projected Risk scores decreasing from 60.24 (unemployed) to 49.66 (part-time) and to 38.36 (full-time employed), with the Overall PRHT Composite of 57.15 (unemployed), 50.46 (part-time), 44.70 (full-time).

TABLE 5
Summary of Multivariate Analysis of Variance
on PRHT Inventory Subscales Scores using
Voter Registration as the Independent Variable (N = 261)^{†}

| | Biology Course Section | | | |
|---|---|---|---|---|
| | Unemployed | Part-Time | Full-Time | |
| Subscale^{†} | (n = 53) | (n = 101) | (n = 107) | F-ratio |
| Police Peril | 73.19 | 70.60 | 62.61 | 11.99** |
| | (14.61) | (13.48) | (12.61) | |
| Community Disrepair | 77.78 | 75.33 | 66.85 | 4.31* |
| | (24.57) | (24.89) | (24.89) | |
| Community Violence | 61.48 | 51.85 | 47.82 | 5.93** |
| | (22.51) | (22.39) | (23.89) | |
| Community Crime | 70.33 | 60.96 | 54.27 | 6.68** |
| | (23.28) | (23.59) | (28.14) | |
| Housing Instability | 36.33 | 27.95 | 27.47 | 5.87 |
| | (20.64) | (16.79) | (13.50) | |
| Violence Experience | 21.89 | 17.26 | 17.26 | 8.37** |
| | (8.29) | (7.42) | (6.58) | |
| Projected Risk | 60.24 | 49.66 | 38.36 | 18.74** |
| | (21.04) | (22.08) | (20.20) | |
| PRHT Composite^{††} | 57.15 | 50.46 | 44.70 | 16.47** |
| | (12.97) | (13.03) | (12.21) | |

*p < .05; **p < .01
^{†}Parenthesized value reflects group standard deviation.
^{††}Result based on Univariate Analysis of Variance method.

The system's multivariate analysis capabilities thus provide tangible intervention outcomes. For example, these capabilities enable targeted intervention programs based on employment status, with documented risk reductions (e.g., Projected Risk scores decreasing from 60.24 to 38.36 for individuals gaining full-time employment), customized intervention strategies accounting for demographic factors such as church attendance and voter registration, and measurable risk reduction simultaneously across multiple domains.

Intervention threshold calculations represent dynamic numerical boundaries that trigger specific intervention recommendations. These thresholds are automatically adjusted based on: (i) statistical significance of measured intervention outcomes; (ii) correlation strength between risk factors; (iii) geographic distribution patterns of risk indicators; and (iv) demographic segmentation analysis results.

Further, path coefficients quantify the direct and indirect effects between risk variables in the system's statistical models, measuring how changes in one risk factor influence others through both direct relationships and mediating variables. These coefficients are continuously updated based on new intervention outcomes and survey responses.

The risk determination engine 118 maintains the foregoing technical measurements in a real-time geospatial database through specialized data structures that preserve statistical relationships while enabling dynamic updates based on new data inputs and intervention results.

All statistical analyses are performed by the system in real-time as new survey data and intervention outcomes are received. The system thus continuously: (i) processes incoming survey data through the validated factor analysis model; (ii) calculates updated path coefficients and mediation effects; (iii) maintains correlation matrices for all risk variables; (iv) performs multivariate analyses to segment populations; (v) recalibrates risk assessment weights based on intervention outcomes; and (vi) generates statistically-validated risk scores and recommendations. The statistical methodologies are preferably implemented through machine learning algorithms that automatically update weights and risk calculations based on statistical significance thresholds (p<0.05 or p<0.01) and model fit indices to maintain predictive accuracy.

In an exemplary configuration, those machine learning algorithms process the statistical data and generate risk assessments. In this regard, those algorithms may use, for example, a neural network architecture configured to process confirmatory factor analysis results by analyzing relationships between community risk factors and personal risk factors, calculate and continuously update path coefficients that quantify direct and indirect effects between variables, and generate risk prediction quotients based on weighted statistical analysis of multiple risk segments. Such a neural network may maintain separate processing layers for geographic risk distribution analysis, demographic segment classification, intervention outcome prediction, and risk factor correlation calculation. Further, those algorithms may employ decision tree algorithms to classify risk levels based on multiple input variables, determine optimal intervention thresholds, and generate branching intervention recommendations based on risk profiles. Still further, such algorithms may use random forest methods to validate risk predictions across multiple decision paths, identify key risk factors driving negative outcomes, and reduce overfitting in risk assessment models. The system preferably maintains model accuracy through continuous retraining on new survey data, dynamic adjustment of neural network weights based on intervention outcomes, and regular recalibration of decision boundaries using validated statistical relationships. Those machine learning algorithms may store and update their parameters within the real-time geospatial database, maintaining links between neural network weight matrices, decision tree split points, random forest configurations, and statistical reliability metrics. Overall, such multi-algorithm approach may enable the system described herein to process complex relationships between risk factors and generate statistically validated risk assessments. Further, this approach may provide evidence-based intervention recommendations and may continuously improve prediction accuracy through automated learning.

This real-time statistical processing provides practical risk mitigation, allowing intervention partners to continuously monitor risk levels across populations and identify emerging risk patterns before they become critical. Likewise, such real-time statistical processing allows those intervention partners to adjust intervention strategies based on measured outcomes, and to document intervention success through quantifiable metrics. The statistical methodologies directly support intervention success by identifying specific factors requiring immediate attention, recommending evidence-based intervention products, tracking intervention effectiveness through numeric indicators, automatically adjusting recommendations based on documented outcomes, and providing measurable proof of risk reduction.

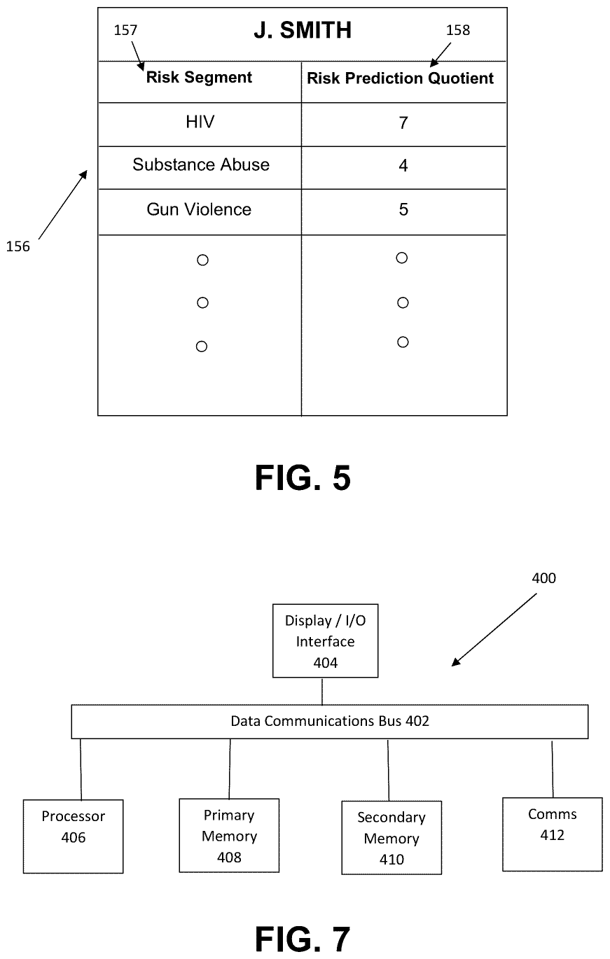

As shown in FIG. 5, the predictive quotient matrix 156 for the risk population member 152 may include multiple risk segments 157 for which user perception data were collected in perceived risk hierarchy inventory 154, and an associated, calculated, numerical risk prediction quotient 158 for each risk segment 157. Varying weights may be applied by risk determination engine 118 to the risk segments 157 in the risk population member's predictive quotient matrix 156 based on discovered interrelationships between perceived risks and actual risks experienced by individuals that are similarly situated to the surveyed member 152 (e.g., of similar race, neighborhood, age, or other demographic characteristics). The weighted values of the risk population member's risk predictive quotient matrix 156 may then be run through a combination or menu of statistical tests and/or methods as described above applied by risk determination engine 118 to single out the greatest areas of risk and those interrelationships that would benefit from intervention, such as through application of intervention products and tools 119. The risk population member's risk portfolio may thus provide a ranking and prioritizing of risk outcomes based on a set of values, beliefs/attitudes and knowledge, and the input data received from perceived risk hierarchy inventory 154 may be used to calculate the level of risk of participation in certain high-risk behaviors. More particularly, the risk determination engine 118 may analyze the risk population member's risk portfolio 116 to generate a value indicative of the risk that such risk population member 152 will engage in the identified risk-associated behavior or activity. A composite quotient based on all high-risk behaviors may also be used to identify a risk population member. Further, where such value for a given risk-associated behavior exceeds a predetermined value that may be selected and adjusted by a system administrator for varying risk populations, predictive risk assessment unit 110 may assign one or more intervention products 119 to the member's risk portfolio 116, which intervention product has been determined to reduce the risk of the member engaging in such risk-associated behavior.

Preferably, a database of risk factors and associations 120 is provided that defines interrelationships among the various risk factors that are analyzed by risk determination engine 118, which interrelationships are preferably expressed in an index that can be displayed in tabular form or graphically, as in (by way of non-limiting example) a Geographical Information System (GIS). Such database of risk factors and associations 120 may be updated through ongoing direct contact with risk population members 152. More particularly, through direct contact with residents of multiple neighborhoods, inter-relationships between variations of perceived risks versus profile data may be used to define and continuously update thresholds that can be displayed in a tabular fashion, in a heat map, or in such other visual presentation as may be preferred by those skilled in the art.

Preferably, a real-time geospatial database serves as the system's primary data store, integrating the multiple specialized data components described above of survey template repository 114 (which maintains survey structures and questions), individual risk portfolio storage 116 (which maintains risk assessment data linked to geographic and demographic factors), and risk factor relationship definitions and indexing 120 (which tracks statistical correlations and associations). Such integrated database structure enables real-time processing of risk assessment data while maintaining distinct functional components for different types of data management and analysis. Generally, the real-time geospatial database is a dynamically updated database system that stores and processes multiple types of location-based risk assessment data, including digital perceived risk data from survey devices, real-time public data containing risk factors associated with geographic locations, and statistical interrelationships between risk variables. The database maintains correlation matrices tracking relationships between risk segments and enables generation of visual heat maps displaying geographical risk distributions. It continuously updates with new survey responses, intervention outcomes, and recalibrated risk predictions, while storing publicly accessible data about social and cultural environmental factors associated with specific locations. The database integrates with the system's machine learning capabilities to maintain statistical reliability coefficients, population segmentation data, and intervention effectiveness metrics across different geographic areas. This geospatial database structure enables real-time analysis of risk patterns and intervention outcomes across different demographic segments and geographic locations.

With continuing reference to FIG. 1, predictive assessment unit 110 also provides an intervention engine 122 preferably capable of (i) determining an intervention product or tool 119 that has been determined to mitigate the risk of a risk population member 152 engaging in a harmful behavior and assigning such intervention product or tool 119 to the risk population member's individual risk portfolio 116, and (ii) modifying the determination of what intervention product or tool 119 may mitigate a given risk based upon a measured effectiveness of an applied intervention product or tool 119 by an intervention partner 180. More particularly, intervention engine 122 analyzes a risk population member's individual risk portfolio 116 to automatically assign an intervention from intervention products/tools 119, such as (by way of non-limiting example) one or more interventions 119 that have been determined as helpful to minimize risk and prevent future disabling social phenomena and/or other negative incident. The risk population member's risk predictive quotient matrix 156 (FIG. 5) is used by the risk determination engine 118 to determine the level of associated risk based on the risk prediction quotient 158 (e.g., high-risk critical, high-risk non-critical, moderate-risk, low-risk, etc.), and uses that level of associated risk to select and assign intervention products/tools 119 in an effort to reduce that risk. Optionally, data collected from multiple neighborhoods and the interrelationships between various perceived risks versus risk population member risk portfolio (which as mentioned above may be used to generate a heat map or table) may also be used to select and assign intervention products/tools 119 to similarly-situated risk population members 152, which may be particularly helpful where individual members 152 have not been surveyed and/or had an individual risk portfolio 116 established by predictive risk assessment unit 110.

With further reference to FIG. 1, intervention partner computers 180 are also preferably in remote data communication with predictive risk assessment unit 110. Intervention partner computers 180 engage with predictive risk assessment unit 110 through an intervention partner user interface 124, and after authentication (such as by password protected login or such other authentication method as may be selected by those skilled in the art) may select an individual risk portfolio 116 for a member of risk population 152 that the associated intervention partner is serving (e.g., e.g., educational institutions, community centers, hospitals, counselors, physician's offices, etc.). Intervention partner computers 180 may receive one or more intervention products 119 that are assigned to the individual risk portfolio 116 of their respective risk population member 152, such that an intervention partner user of intervention partner computer 180 may oversee the administration of such intervention product/tool 119. Based upon that intervention partner user's observations of risk population member 152 after administration of the associated intervention product/tool 119, the intervention partner user may transmit from intervention partner computer 180 data reporting the success of administration of the respective intervention product/tool 119 (e.g., a numeric score indicating a designated level of success, which may vary from behavior to behavior) in mitigating the particular dangerous or harmful behavior or activity, and the success score for the applied intervention product/tool 119 may be assigned to the respective risk population member's individual risk portfolio 116.

Based upon the results of the intervention product/tool 119 applied to the respective risk population member 152 (as evidenced by the numeric score assigned to such intervention product/tool 119), and as part of a feedback process, intervention engine 122 may make further adjustments in weightings applied to the risk segments 157 in the risk population member's predictive quotient matrix 156. Questions in the survey applied by survey device 150 may be added or subtracted to change particular weights assigned to various elements of the digital perceived risk hierarchy inventory 154, and different statistical methods or algorithms may be applied to the analysis by intervention engine 122 as detailed above. Moreover, as part of an ongoing data collection effort, predictive risk assessment unit 110 may carry out further iterations to reflect the new line of questions that will be posed to the risk population. Still further, as part of the feedback process, the results of the adjustments in weighting made by intervention engine 122 may be compared to actual results of a given intervention product/tool 119 to validate the risk index/probability assessment.

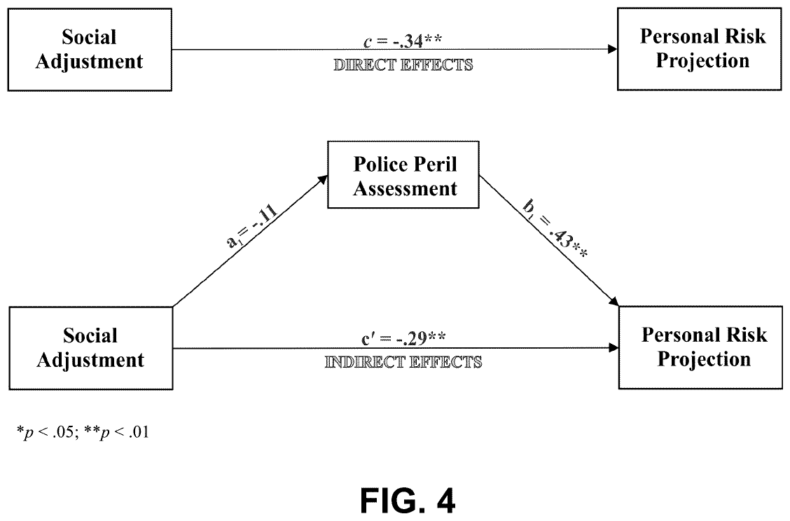

The statistical relationships and coefficients illustrated in FIGS. 2 to 4 may be dynamically updated through the system's automated feedback process. Specifically, the confirmatory factor analysis relationships shown in FIG. 2, including the beta coefficients between Community Risk Factors and their correlation, may be continuously recalculated as new survey data and intervention outcomes are processed. Similarly, the path analysis model shown in FIG. 3 maintains dynamic path coefficients that may be automatically adjusted based on intervention effectiveness data. The direct effects of Community Risk Factors, mediating effects of Social Adjustment, impact of Police Peril Assessment, and influence of Personal Risk Factors may all be subject to automatic recalibration when the system identifies statistically significant changes in risk relationships. Further, the mediation analysis illustrated in FIG. 4, showing relationships between Social Adjustment and Personal Risk Projection through Police Peril Assessment, may likewise be updated through the system's machine learning algorithms. Both direct effects and indirect effects may be recalculated as the system processes new data about these relationships.

These dynamic updates may be maintained while preserving statistical validity through continuous reliability calculations and correlation analysis. The system may automatically trigger recalibration when statistical relationships change beyond predetermined thresholds, ensuring that the models shown in FIGS. 2-4 accurately reflect current risk patterns and intervention effectiveness.

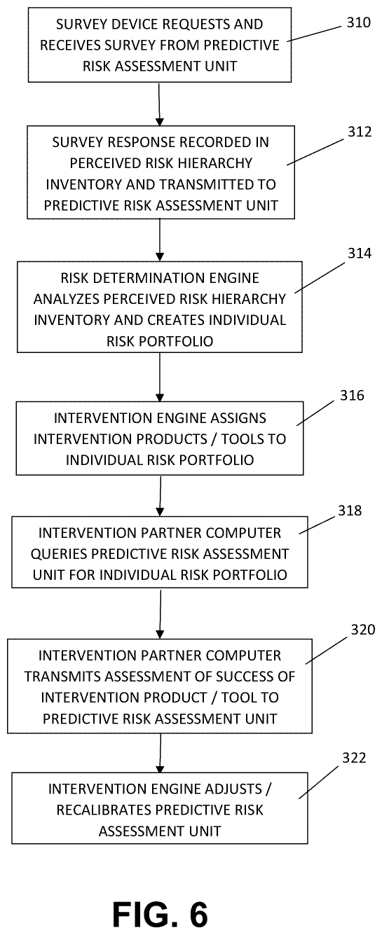

Next, FIG. 6 is a schematic view of a computer-implemented process for predictive risk assessment and intervention in accordance with further aspects of an embodiment. At step 310, an authenticated user of survey device 150 causes survey device 150 to request a survey 114 from predictive risk assessment unit 110, which survey 114 is then transmitted to survey device 150 for administration to a risk population member 152. Next at step 312, the risk population member's responses to survey 114 are recorded in a perceived risk hierarchy inventory 154 which is transmitted from survey device 150 to predictive risk assessment unit 110. At step 314, risk determination engine 118 analyzes the perceived risk hierarchy inventory 154 and establishes an individual risk portfolio 116 for the respective risk population member 152, which includes a risk predictive quotient matrix assigning a numeric risk prediction quotient 158 to each of one or more risk segments 157. At step 316, intervention engine 122 assigns one or more intervention products/tools 119 to the individual risk portfolio 116 for that risk population member, which intervention products/tools 119 are calculated as being able to mitigate the risk of that risk population member 152 engaging in a dangerous behavior or activity. At step 318, an intervention partner computer 180 queries predictive risk assessment unit 110 to obtain the individual risk portfolio 116 for the risk population member 152 that they are servicing and thereafter may administer the intervention products/tools 119 associated with that member's individual risk portfolio 116. Next, at step 320, the intervention partner computer 180 transmits to predictive risk assessment unit 110 an assessment of the success of application of the intervention product/tool 119 in mitigating the risk of the risk population member engaging in a particularly dangerous or harmful activity. At step 322, intervention engine 122 evaluates the assessment received from intervention partner computer 180 and preferably adjusts and/or recalibrates the weights assigned to differing risk segments in the risk population member's risk predictive quotient matrix, and/or modifies the questions presented by survey device 150 and statistical methods applied by risk determination engine 118 to further refine the risk population member's individual risk portfolio 116.

Those skilled in the art will recognize that each of predictive risk assessment unit 110, survey device 150, and intervention partners 180 may each take the form of computer system 400 as reflected in FIG. 7, though variations thereof may readily be implemented by persons skilled in the art as may be desirable for any particular installation. In each such case, one or more computer systems 400 may carry out the foregoing methods as computer code.

Computer system 400 includes a communications bus 402, or other communications infrastructure, which communicates data to other elements of computer system 400. For example, communications bus 402 may communicate data (e.g., text, graphics, video, other data) between bus 402 and an I/O interface 404, which may include a display, a data entry device such as a keyboard, touch screen, mouse, or the like, and any other peripheral devices capable of entering and/or viewing data as may be apparent to those skilled in the art. Further, computer system 400 includes a processor 406, which may comprise a special purpose or a general purpose digital signal processor. Still further, computer system 400 includes a primary memory 408, which may include by way of non-limiting example random access memory (“RAM”), read-only memory (“ROM”), one or more mass storage devices, or any combination of tangible, non-transitory memory. Still further, computer system 400 includes a secondary memory 410, which may comprise a hard disk, a removable data storage unit, or any combination of tangible, non-transitory memory. Finally, computer system 400 may include a communications interface 412, such as a modem, a network interface (e.g., an Ethernet card or cable), a communications port, a PCMCIA slot and card, a wired or wireless communications system (such as Wi-Fi, Bluetooth, Infrared, and the like), local area networks, wide area networks, intranets, and the like.

Each of primary memory 408, secondary memory 410, communications interface 412, and combinations of the foregoing may function as a computer usable storage medium or computer readable storage medium to store and/or access computer software including computer instructions. For example, computer programs or other instructions may be loaded into the computer system 400 such as through a removable data storage device (e.g., a floppy disk, ZIP disks, magnetic tape, portable flash drive, optical disk such as a CD, DVD, or Blu-ray disk, Micro Electro Mechanical Systems (“MEMS”), and the like). Thus, computer software including computer instructions may be transferred from, e.g., a removable storage or hard disc to secondary memory 410, or through data communication bus 402 to primary memory 408.

Communication interface 412 allows software, instructions and data to be transferred between the computer system 400 and external devices or external networks. Software, instructions, and/or data transferred by the communication interface 412 are typically in the form of signals that may be electronic, electromagnetic, optical or other signals capable of being sent and received by communication interface 412. Signals may be sent and received using a cable or wire, fiber optics, telephone line, cellular telephone connection, radio frequency (“RF”) communication, wireless communication, or other communication channels as will occur to those of ordinary skill in the art.

Computer programs, when executed, allow processor 406 of computer system 400 to implement the methods discussed herein for predictive risk assessment and intervention according to computer software including instructions.

Computer system 400 may perform any one of, or any combination of, the steps of any of the methods described herein. It is also contemplated that the methods according to the present invention may be performed automatically or may be accomplished by some form of manual intervention.

The computer system 400 of FIG. 7 is provided only for purposes of illustration, such that the invention is not limited to this specific embodiment. Persons having ordinary skill in the art are capable of programming and implementing the instant invention using any computer system.

Further, computer system 400 may, in certain implementations, comprise a handheld device and may include any small-sized computing device, including by way of non-limiting example a cellular telephone, a smartphone or other smart handheld computing device, a personal digital assistant, a laptop or notebook computer, a tablet computer, a hand held console, an MP3 player, or other similarly configured small-size, portable computing device as may occur to those skilled in the art.

As explained above, the system of FIG. 1 may, in an exemplary configuration, be implemented in a cloud computing environment for carrying out the methods described herein. That cloud computing environment uses the resources from various networks as a collective virtual computer, where the services and applications can run independently from a particular computer or server configuration making hardware less important. The cloud computer environment includes at least one survey device 150 operating as a client computer. The client computer may be any device that may be used to access a distributed computing environment to perform the methods disclosed herein, and may include (by way of non-limiting example) a desktop computer, a portable computer, a mobile phone, a personal digital assistant, a tablet computer, or any similarly configured computing device. That client computer preferably includes memory such as RAM, ROM, one or more mass storage devices, or any combination of the foregoing. The memory functions as a computer readable storage medium to store and/or access computer software and/or instructions.

That client computer also preferably includes a communications interface, such as a modem, a network interface (e.g., an Ethernet card), a communications port, a PCMCIA slot and card, wired or wireless systems, and the like. The communications interface allows communication through transferred signals between the client computer and external devices including networks such as the Internet and a cloud data center. Communication may be implemented using wireless or wired capability, including (by way of non-limiting example) cable, fiber optics, telephone line, cellular telephone, radio waves or other communications channels as may occur to those skilled in the art.

Such client computer establishes communication with the one more servers via, for example, the Internet, to in turn establish communication with one or more cloud data centers that implement predictive risk assessment and intervention system 100. A cloud data center may include one or more networks that are managed through a cloud management system. Each such network includes resource servers that permit access to a collection of computing resources and components of predictive risk assessment and intervention system 100, which computing resources and components can be invoked to instantiate a virtual computer, process, or other resource for a limited or defined duration. For example, one group of resource servers can host and serve an operating system or components thereof to deliver and instantiate a virtual computer. Another group of resource servers can accept requests to host computing cycles or processor time, to supply a defined level of processing power for a virtual computer. Another group of resource servers can host and serve applications to load on an instantiation of a virtual computer, such as an email client, a browser application, a messaging application, or other applications or software.

The cloud management system may comprise a dedicated or centralized server and/or other software, hardware, and network tools to communicate with one or more networks, such as the Internet or other public or private network, and their associated sets of resource servers. The cloud management system may be configured to query and identify the computing resources and components managed by the set of resource servers needed and available for use in the cloud data center. More particularly, the cloud management system may be configured to identify the hardware resources and components such as type and amount of processing power, type and amount of memory, type and amount of storage, type and amount of network bandwidth and the like, of the set of resource servers needed and available for use in the cloud data center. The cloud management system can also be configured to identify the software resources and components, such as type of operating system, application programs, etc., of the set of resource servers needed and available for use in the cloud data center.

In accordance with still further aspects of an embodiment of the invention, a computer program product may be provided to provide software to the cloud computing environment. Computer products store software on any computer useable medium, known now or in the future. Such software, when executed, may implement the methods according to certain embodiments of the invention. By way of non-limiting example, such computer usable mediums may include primary storage devices (e.g., any type of random access memory), secondary storage devices (e.g., hard drives, floppy disks, CD ROMS, ZIP disks, tapes, magnetic storage devices, optical storage devices, MEMS, nanotech storage devices, etc.), and communication mediums (e.g., wired and wireless communications networks, local area networks, wide area networks, intranets, etc.). Those skilled in the art will recognize that the embodiments described herein may be implemented using software, hardware, firmware, or combinations thereof.

The cloud computing environment described above is provided only for purposes of illustration and does not limit the invention to this specific embodiment. It will be appreciated that those skilled in the art are readily able to program and implement the invention using any computer system or network architecture.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. A computer-implemented method for predictive risk assessment and intervention, comprising:
providing a predictive risk assessment unit having a processor executing a machine learning algorithm and a memory storing a real-time geospatial database;
receiving, at said predictive risk assessment unit, digital survey data from a remote survey device comprising risk perception data and demographic data associated with a human risk population member;
performing, by said processor, confirmatory factor analysis on said digital survey data to identify community risk factors and personal risk factors;
executing, by said processor, path analysis modeling to:
calculate direct effect coefficients between said risk factors;
determine mediating effects between social adjustment factors and risk projections; and
generate risk assessment scores based on said effect coefficients;
maintaining, in said real-time geospatial database, a correlation matrix tracking statistical relationships between risk variables;
automatically generating, by said processor, an individual risk portfolio for said human risk population member comprising:
risk prediction quotients for multiple risk segments;
weighted statistical analysis of said risk prediction quotients; and
recommended intervention products based on said weighted statistical analysis;
transmitting said individual risk portfolio to an intervention partner computer;
receiving intervention outcome data comprising numeric indicators of intervention success;
automatically recalibrating said machine learning algorithm by:
updating path coefficients based on said intervention outcome data;
adjusting risk assessment score calculations; and
modifying intervention product recommendations; and
generating a modified individual risk portfolio based on said recalibrated machine learning algorithm.

2. The method of claim 1, wherein said processor maintains statistical reliability through calculation of Cronbach's alpha coefficients for risk assessment subscales comprising:
behavioral risk indicators;
environmental risk factors;
social interaction metrics;
geographic risk elements;
residential stability measures;
personal safety indicators; and
risk projection factors.

3. The method of claim 1, wherein said processor performs multivariate analysis of variance to:
segment populations based on socioeconomic indicators;
identify statistically significant differences in behavioral patterns;
customize intervention recommendations based on demographic characteristics; and
generate risk mitigation strategies across multiple behavioral domains.

4. The method of claim 1, further comprising:
generating visual heat maps displaying geographical risk distributions; and
updating said heat maps in real-time based on newly received survey data and intervention outcomes.

5. The method of claim 1, wherein said processor executes real-time model updates by:
maintaining statistical relationships between community and personal risk factors;
updating risk prediction quotients based on newly received data; and
adjusting intervention thresholds based on validated outcomes.

6. The method of claim 5, wherein said machine learning algorithm processes new data by:
integrating real-time survey responses with existing risk factor correlations;
validating factor analysis results through statistical reliability calculations; and
updating population segmentation based on multivariate analysis of demographic data.

7. The method of claim 6, wherein said machine learning algorithm implements automated feedback processing by:
analyzing intervention success indicators across demographic segments;
adjusting risk assessment calculations based on validated outcomes; and
modifying intervention recommendations based on success patterns.

8. The method of claim 7, wherein recalibrating said machine learning algorithm comprises:
updating correlation coefficients based on intervention outcome data;
adjusting risk segment weights based on intervention success rates; and
modifying path analysis coefficients to reflect new risk relationships.

9. The method of claim 8, wherein said processor maintains model accuracy by:
continuously validating statistical relationships through reliability calculations;
automatically adjusting risk assessments when correlations change beyond thresholds; and
recalibrating intervention recommendations based on updated risk profiles.

10. A computer system for predictive risk assessment and intervention, comprising:
a predictive risk assessment unit comprising:
a processor executing a machine learning algorithm; and
a memory storing a real-time geospatial database;
a remote, portable survey device in data communication with said predictive risk assessment unit, configured to:
display digital survey interfaces;
collect risk perception data and demographic data from a human risk population member; and
transmit collected data to said predictive risk assessment unit; and
an intervention partner computer in data communication with said predictive risk assessment unit;
wherein said processor is configured to:
perform confirmatory factor analysis on received survey data to identify community risk factors and personal risk factors;
execute path analysis modeling to:
calculate direct effect coefficients between said risk factors;
determine mediating effects between social adjustment factors and risk projections; and
generate risk assessment scores based on said effect coefficients;
maintain, in said real-time geospatial database, a correlation matrix tracking statistical relationships between risk variables;
automatically generate an individual risk portfolio comprising:
risk prediction quotients for multiple risk segments;
weighted statistical analysis of said risk prediction quotients; and
recommended intervention products based on said weighted statistical analysis;
transmit said individual risk portfolio to said intervention partner computer;
receive intervention outcome data comprising numeric indicators of intervention success;
automatically recalibrate said machine learning algorithm by:
updating path coefficients based on said intervention outcome data;
adjusting risk assessment score calculations; and
modifying intervention product recommendations; and
generate a modified individual risk portfolio based on said recalibrated machine learning algorithm.

11. The system of claim 10, wherein said processor maintains statistical reliability by:
executing Cronbach's alpha coefficient calculations for risk assessment subscales by:
analyzing response pattern consistency across multiple survey inputs;
calculating internal consistency metrics for each risk indicator category;
validating statistical significance of reliability measures;
storing calculated coefficients in said real-time geospatial database; and
performing continuous reliability validation through:
comparing new survey responses against existing reliability metrics;
identifying statistically significant deviations in response patterns;
adjusting coefficient calculations based on validated changes; and
updating stored reliability measures in real-time.

12. The system of claim 10, wherein said processor performs multivariate analysis by:
executing statistical computations to:
calculate variance matrices across demographic segments;
determine statistical significance of behavioral differences;
identify correlation patterns between risk indicators; and
generate weighted risk factor relationships; and
implementing automated population segmentation through:
real-time processing of demographic identifier data;
statistical clustering of response patterns;
dynamic updating of segment definitions; and
continuous validation of segment boundaries.

13. The system of claim 12, wherein said processor executes real-time model updates by:
maintaining risk factor relationship definitions and statistical correlations in a risk factor indexing component of said real-time geospatial database;
updating individual risk portfolios stored in said real-time geospatial database based on newly received survey response data;
integrating real-time public data with stored risk assessments to adjust intervention thresholds; and
linking updated risk assessments to geographic locations and demographic segments.

14. The system of claim 13, wherein said processor executes said machine learning algorithm to process new data by:
retrieving survey templates from a survey repository component of said real-time geospatial database;
validating new survey responses against existing risk factor correlations;
updating population segmentation data through integrated analysis of demographic factors and geographic distributions; and
storing processed results in corresponding individual risk portfolio components while maintaining geographic and demographic relationships.

15. The system of claim 14, wherein said processor implements automated feedback processing by:
storing intervention outcome data in said real-time geospatial database with maintained links to:
corresponding individual risk portfolios;
geographic location identifiers; and
demographic segment classifications;
analyzing intervention effectiveness through:
calculating success metrics across linked demographic segments;
evaluating geographic distribution patterns of outcomes; and
identifying statistically significant outcome variations;
automatically updating risk assessments by:
modifying individual risk portfolio components based on validated outcomes;
adjusting risk factor correlations in the indexing component; and
recalibrating intervention recommendations based on success patterns.

16. The system of claim 15, wherein said processor recalibrates said machine learning algorithm by:
analyzing intervention outcome data stored in said real-time geospatial database to:
update correlation coefficients between risk factors;
modify demographic segment definitions; and
adjust geographic risk distribution patterns;
integrating recalibrated values across database components to:
update risk factor relationship definitions;
modify intervention threshold calculations; and
adjust population segmentation parameters; and
storing updated algorithm parameters in said real-time geospatial database with maintained links to:
source intervention outcomes;
affected geographic regions; and
impacted demographic segments.

17. The system of claim 16, wherein said processor maintains model accuracy by:
continuously validating data relationships across integrated database components through:
statistical reliability calculations on stored risk assessments;
correlation analysis between geographic risk patterns; and
intervention effectiveness comparisons across segments;
automatically triggering model updates when:
reliability metrics exceed predetermined thresholds;
geographic risk patterns show significant shifts; and
intervention outcomes indicate effectiveness changes;
maintaining data consistency by:
synchronizing updates across database components;
preserving relationship links between data elements; and
validating data integrity after modifications.
